(19) United States
(12) Patent Application Publication
(10) Pub. No.: US 2010/0035294 A1
(43) Pub. Date: Feb. 11, 2010

(54) SYSTEM TO ASSESS THE BIOLOGICAL ACTIVITY OF CHEMOATTRACTANTS

(75) Inventor: Andreas J. Kungl, Graz (AT)

Correspondence Address: PROSKAUER ROSE LLP, One International Place, Boston, MA, 02110, US

(73) Assignee: Protaffin Biotechnologie AG, graz (AT)

(21) Appl. No.: 12/446,838

(22) PCT Filed: Oct. 30, 2007
(86) PCT No.: PCT/AT2007/000495

§ 371 Date: Apr. 23, 2009

(30) Foreign Application Priority Data
Oct. 30, 2006 (AT) A 1821/2006

(51) Int. Cl. C12Q 1/02; C12M 1/34

(52) U.S. Cl. 435/29; 435/287.9

(57) ABSTRACT

The present invention relates to a system for measuring the biological activity of chemoattractants comprising at least two units separated by a semipermeable carrier wherein biologically active carbohydrate structures, preferably glycosaminoglycan (GAG) structures, are immobilized on the surface of said carrier. According to the invention, this system can be used for fast and economic measurement of the degree of cell mobility and chemotactic activity.

The present invention relates to a system for measuring the biological activity of chemoattractants.

The study of cellular behaviour upon external stimuli, and here especially cell movement and differentiation, are prevalent throughout contemporary biological research. Generally, this research involves exposing cells to various physical/chemical/biological stimuli and monitoring/quantifying the cells' response. By exogenously stimulating viable cells, information on basic principles of cellular metabolism as well as on the mode of effector action is obtained. In the following, special emphasis will be placed on exogenously induced chemotaxis.

When a cell is exposed to physical/chemical/biological stimuli, its response deserves special consideration particularly when developing and evaluating therapeutic compounds and their pharmacological efficacy. Especially in the fields of oncology and inflammation research, the impact of anti-cancer/anti-inflammation drugs and drug candidates on cell migration needs to be considered. Usually, these types of studies are carried out, in a first approach, ex vivo using either immortalised cell lines or patient-derived cell preparations. In addition to chemotactic parameters, these assays can also provide insight into the processes of tissue regeneration, wound healing, inflammatory diseases, autoimmune diseases, and many other degenerative diseases and conditions.

In vitro cell migration assays are typically used in conducting this kind of research. Commercially available devices for performing such assays are often based on or employ a Boyden chamber. This is a vessel partitioned by a semi-permeable membrane into two distinct, super-imposed units: Unit 1 (lower unit) and Unit 2 (upper unit). The Boyden chamber is used by placing a migratory/chemotactic molecule into Unit 1 and the cells to be studied into Unit 2. After a sufficient incubation period, the cells may be fixed, stained, and counted to study the effects of the stimulus on cell migration across the membrane (Falk et al. (1980), J. Immunol. Methods 33: 239-247).

Alternatively, trans-well assays can be used in a set-up similar to the Boyden chamber whereby the separation of the two (migratory cells- and chemoattractant-containing) units is accomplished not only by a membrane but by (endothelial) cell-coated membranes (Weber et al. (1997) J. Immunol. 159:3968-75.). This set-up has several disadvantages. For instance, assays employing transwells require a labor-intensive protocol that is not easily adaptable to high-throughput screening and processing. The counting of cells, which is often done manually using a microscope, is a time-consuming, tedious, and expensive process. Furthermore, cell counting is also subjective and involves statistical approximations. Specifically, due to the time and expense associated with examining an entire filter, only representative areas, selected at random, may be counted, and, even when these areas are counted, if a cell has only partially migrated through the filter, the scientist/technician must, nevertheless, exercise his or her judgement when accounting for such a cell. In light of the multiple samples required for each test, in addition to the positive and negative controls required to obtain reliable data, a single chemotaxis assay can require dozens of filters, each of which needs to be individually examined and counted.

Monitoring cell migration and differentiation is important for the understanding of numerous biological functions, both physiological and pathological. For example, the study of tissue regeneration and wound healing, as well as the study of inflammation, autoimmune diseases and other degenerative diseases, all involve the analysis of cell movement, either spontaneous or in response to chemotactic factors or other cellular signals. Further, for investigating the treatment of various abnormal tissue functions or diseases, scientists must analyze the effects of potential therapies on cell movement in cell culture before proceeding to clinical studies.

As the migratory behaviour of cells has potential implications for the development of certain therapeutics, a better and more sophisticated in vitro system is needed for screening and quantifying the effects of drugs and drug candidates on cell motility and migration. Especially the consideration of target cells/tissues, to where migratory cells are attracted, deserves more attention. The interaction of chemotactic cytokines, the so-called chemokines, with glycosaminoglycan (GAG) structures on the surface of endothelial cells is a crucial step in the inflammation cascade (Proudfoot et al. (2003) Proc. Natl. Acad. Sci. U.S.A. 100: 1885-90). During this process, chemokines are sequestered on these glycan structures to form a solid-phase chemotactic gradient by which migratory cells like leukocytes are attracted. Therefore, assay systems wherein the biological activity of chemokines in the presence of biologically active GAG molecules can be measured, are very valuable. For this purpose, the membranes of conventional Boyden chambers can be covered with target cells, such as endothelial cells, and leukocyte transmigration can be determined. These so-called trans-well assays are, however, highly time- and cost-intensive and do not provide the user with standardized results due to cell variation. So there is a high demand in getting a system where the cost-intensive procedure can be avoided, which provides highly reproducible results, and which can be set-up for high-throughput compound evaluation.

It is an object of this invention to provide a system for a simple, economical, effective, and specific measurement of the biological activity of chemoattracting substances like chemokines.

Accordingly, the present invention provides a system to assess the biological activity of chemoattractants comprising at least a first unit and a second unit separated by a semipermeable carrier, characterized in that biologically active carbohydrate structures are immobilized on the surface of said carrier. Thereby the cell surface of target endothelial vessel cells in vivo are more appropriately mimicked using this novel assay than in the conventional (modified) Boyden chamber which does not include the relevant binding interactions between proteins and carbohydrates. In addition, the novel assay according to the present invention has the additional benefit of a much easier and faster experimental set-up and throughput than the trans-well assay.

In a preferred embodiment of the invention, glycosaminoglycan structures, e.g. GAGs can be immobilized on the carrier surface to measure the biological activity of chemokines.

One unique feature of the present invention is the use of a semipermeable carrier onto which various biologically active carbohydrate structures can be immobilized. These include N- and O-linked glycan-derived molecules such as the high mannose, the complex, and the hybrid type (sialylated or fucosylated), more specifically Lewis^{a/x}, Lewis Y, SialylTn, (either sialylated or unsialylated), preferably glycosaminoglycan (GAG) structures. This carrier is highly advantageous over the trans-well approach since immobilizing chemically well-defined structures is fast and easily reproducible and thus provides the opportunity for standardized measurements of the biological activity for various chemoattractants. The GAG structures can be any GAGs known in the art (as reviewed in “Conformation of Carbohydrates” by V. S. R. Rao, P. K. Qasba, P. V. Balaji, R. Chandrasekaran, 1998, harwood academic publishers, pp: 162-166). Preferably, these GAGs are heparan sulfate, heparin, chondroitin sulfate, keratan sulfate, dermatan sulfate or hyaluronic acid. The GAGs can be naturally derived, either from healthy tissues or tissues with a certain pathological phenotype with preferably original (unmodified) chain length or size-fractionated by chemical or biochemical means. Alternatively, they can be fully synthetic. Both, naturally derived and chemically synthesised GAGs can be further chemically modified or substituted, e.g. by substituting sulfate by phosphate groups, by introducing hydrophobic substituents, by removing N-acetyl groups and other means well known in the art (as detailed in “Carbohydrates in Chemistry and Biology” by B. Ernst, G. W. Hart, P. Sinay (Eds.), 2000, Wiley-VCH Verlag).

In a further embodiment the GAG is activated GAG. Activation can occur by any methods known in the art (for review see Casu et al., 2002, Seminars Thromb. Hemostasis 28: 335-342 and Fernandez et al., 2006, Carbohydrate Res. 341: 1253-1265) for example coupling GAG via free primary amines (NH₂), via acetyl groups, sulphate groups or hydroxyl groups onto the carrier.

The biologically active carbohydrates, e.g. GAG structures, can be either covalently or non-covalently immobilized on the carrier, for example via affinity binding (biotin-streptavidin), ionic/electrostatic or hydrophobic interaction. The immobilization can preferably be made via linker molecules such as aliphatic linkers, carbohydrate linkers, or aromatic linker structures/compounds.

It is envisioned that the semipermeable carrier can be constructed of any suitable porous material. Semipermeable, i.e. selectively permeable, carriers are available in a variety of forms such as sheets, tubes, and hollow fibers that permit selective exchange of materials across the walls.

Any material can be used as long as the pore size of the carrier is sufficient to allow the chemoattractants and their inhibitors to pass the carrier in both directions and to prohibit excessive uninduced cell migration into the lower chamber. Preferably the carrier is a membrane, especially a membrane selected comprising a polycarbonate, polysulfone, polyvinyl or polystyrene structure.

The pore size of the membranes should preferably be ranging from 0.5 to 10 μm diameter, preferably from 2.5 to 7.5 μm, more preferably approx. 5 μm.

The chemoattractants as used according to the present invention can be any chemoattracting substance known in the art. A chemoattractant is a molecule—preferably a protein, still preferably a chemokine—which gives rise to the migration of certain target cells—preferably leukocytes—by establishing a chemotactic gradient along which the target cells can move (see Kehrl, 2006, Immunol. Res. 34: 211-27). The chemotactic gradient is a solid state phase gradient which is established by binding of chemoattractants to specific tissues or vessels or cell surface walls. The biological activity of chemoattractants is mediated via receptor molecules on the target cells which activate the cell after binding to the chemoattractant. Preferably, they are proteins and more preferably they are chemokines, cytokines, growth factors or derivatives or fragments thereof. In a specific embodiment of the present invention, the chemokines are IL-8, RANTES, SDF-1, I-TAC or MCP-1 or derivatives or fragments thereof.

According to the present invention all derivatives and fragments of chemokines are included that still show at least partial or decreased chemoattracting activity in relation to the unmodified or full-length chemokine. More specifically, it can also be a modified chemokine having increased or knocked-out binding affinity to GAGs and/or further inhibited or down-regulated biological activity compared to the respective wild type IL-8. These modified chemokines can also be called dominant-negative chemokines. Examples of such modified proteins are described in detail in WO 05/054285 A.

According to the present invention, the chemoattractant is present in one of the units of the system, preferably in Unit 1. In a preferred embodiment, the chemoattractant is present in a buffer solution, optionally together with stabilising ions and/or detergents. A preferred buffer for testing the chemoattractant activity of chemokines should be in the pH range 5.0-9.0, preferably in the range 6-8, more preferably in the range 6.5-7.5 and should contain a salt concentration>20 mM NaCl, preferably>100 mM NaCl. Additionally, any detergent substance can be used that prevents unspecific chemokine aggregation.

A chemoattractant inhibitor can be added to the other chamber. This can be an antagonist of the chemoattractant receptor on the target cell, or an antibody raised against the chemoattractant receptor or the chemoattractant itself, or a modified chemoattractant, or an antagonist of the cell surface GAGs, or an antibody raised against cell surface GAGs. Inhibition of chemoattractant activity is defined by the reduced migration of target cells in the chemotaxis assay—as expressed by the number of migrated cells—relative to the non-inhibited situation.

For measuring the biological activity, the upper chamber can contain at least one inhibitor of the chemoattractant, cells, media and/or buffer. Inhibitors of chemoattractants according to the present invention can be any known inhibitors useful, for example, they can be GAGs, analogues, fragments and derivatives thereof, and GAGmimetics (see Freeman et al., 2005, J. Biol. Chem. 280: 8842-8849; Barbosa et al., 2004, J. Cell. Sci. 118: 253-264; Ziebell et al., 2001, Chem. Biol. 8: 1081-1094). These are compounds which resemble natural GAGs either structurally or functionally or both. They can be derived by chemical synthesis or by extraction of a natural source or a combination thereof. Typical GAGmimetics, both structurally and functionally, are, for example, the low molecular weight heparins (LMWHs) which are applied as inhibitors of blood coagulation therefore mimicking the task of physiological heparin released from mast cells. GAGmimetics can be any structures that have the same or similar function as naturally occurring GAGs. Alternatively, the chemoattractant inhibitors can be any natural, modified or mutant protein, preferably a natural, modified or mutant chemokine, preferably a dominant negative chemokine as said above. Alternatively, they can be GPCR antagonists (for example the low molecular weight compound Traficet-EN from ChemoCentryx, a CCR9 antagonist, which is currently in an international clinical Phase II trial with over 400 Crohn's disease patients).

According to a specific embodiment, the inventive system comprises two units wherein Unit 1 contains chemokines, Unit 2 contains leukocytes, and wherein the units are separated by a semipermeable carrier having biotinylated heparin immobilized thereto.

The units suitable for the system according to the present invention can be of any material useful for chemotactic assays (according to the invention the terms chamber and unit can be equally used). Preferably the units are composed of glass or synthetic materials, for example polyethylene or polypropylene. Also the dimensions of the units can be altered to fit the advantageous specification. In principle, also the general architecture of a Boyden chamber can be used also for the present invention; this can easily be adapted by the skilled man in the art according to the teachings according to the present invention.

According to the present invention the system can be used for measuring the degree of cell mobility and/or the degree of chemotactic activity. This can be done by placing a GAG-coated membrane between the two units of a Boyden chamber which separates the unit containing the target cells (Unit 2, see FIG. 1) and the unit containing the chemoattractant (Unit 1). This leads, contrary to a conventional Boyden chamber, to a structural change of the chemoattractant after binding to the GAG molecules on the membrane which is a conformational trigger to activate the chemoattractant receptor on the target cells in a way more closely related to the in vivo situation.

The following examples and the figures are describing the invention in more detail without limiting the scope of the invention.

### Figures

FIG. 1: Schematic picture of the modified Boyden chamber with immobilised GAGs on the semipermeable PC membrane

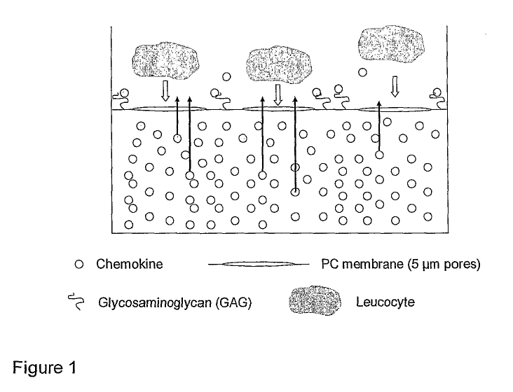

FIG. 2: Results of an IL-8-driven chemotaxis assay on freshly prepared human neutrophils using uncoated and heparin-coated PC membranes at different IL-8 concentrations

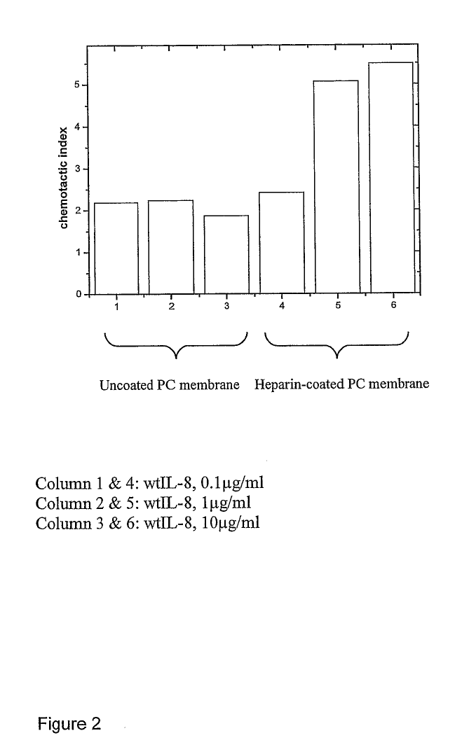

FIG. 3: Results of an IL-8-driven chemotaxis assay on freshly prepared human neutrophils using uncoated, heparin-, heparan sulfate(HS)-, and chondroitin sulfate(CS)-coated PC membranes at different IL-8 concentrations

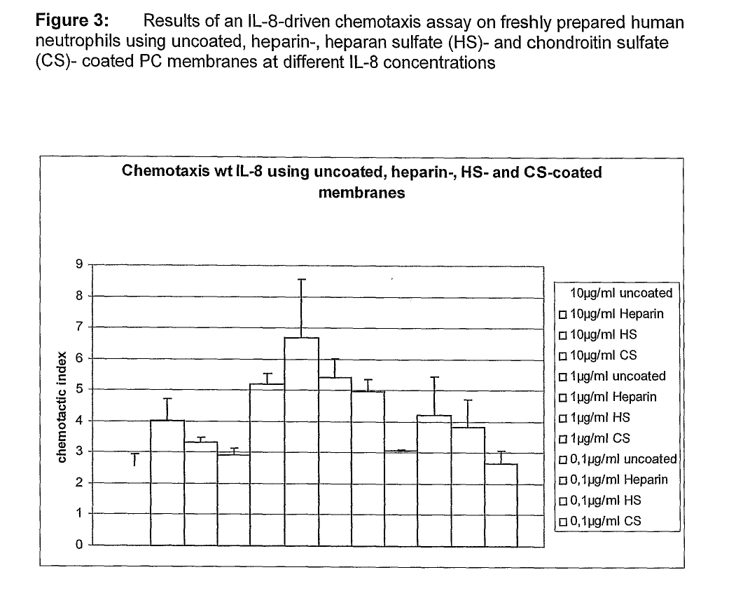

FIG. 4: Results of a RANTES-driven chemotaxis assay on Thp-1 (human monocytic) cells using uncoated, heparin- and heparan sulfate(HS)-coated PC membranes at different RANTES concentrations

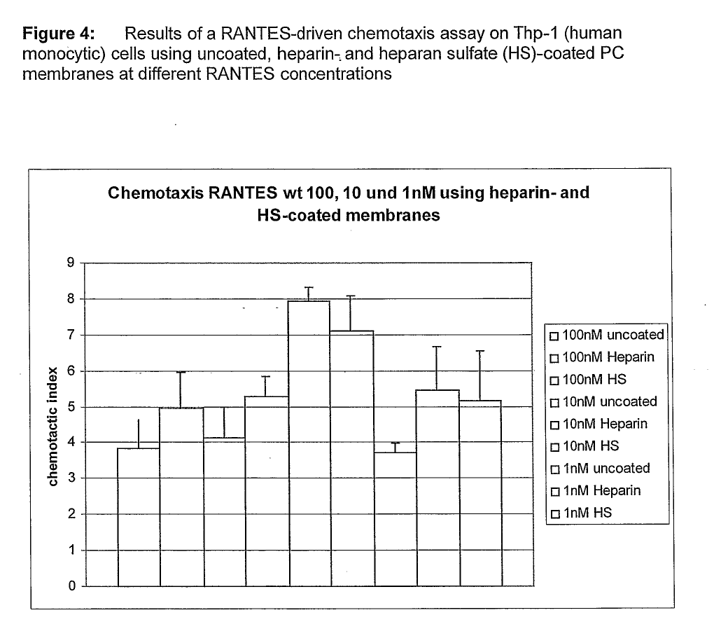

### EXAMPLES

### Chemotaxis Assay in a Heparin-Modified Boyden Chamber

Transfilter chemotaxis of neutrophils in response to IL-8 or RANTES was assayed in a microchemotaxis chamber (Neuroprobes, 48-well Boyden chamber) equipped with a 5 μm PVP-free streptavidin-coated polycarbonate membrane (Neuroprobes) onto which biotinylated heparin was immobilised. PVP-free membranes were found to be more densely coated with streptavidin than PVP-containing membranes. These membranes were subsequently incubated with a 100 μM biotinylated heparin (Sigma) solution in PBS (137 mM NaCl, 2.7 mM KCl, 4.3 mM Na₂HPO₄, 1.4 mM KH₂PO₄, pH 7.3) for 1 hr at room temperature. After thorough washing with PBS, the membranes were used in the chemotaxis assay.

### Cell Preparation:

Briefly, a neutrophil fraction was prepared from freshly collected human blood. This was done by adding a 6% dextran solution to blood (1:2), treated with EDTA for anticoagulation before, which was then left for sedimentation for 45 min. The upper clear cell solution was collected and washed twice with HBSS (0.4 g/l KCl, 0.06 g/l KH₂PO₄, 0.35 g/l NaHCO₃, 8 g/l NaCl, 0.05 g/l Na₂HPO₄). Cells were counted and finally diluted with HBSS at 2 Mio/ml cell suspension, taking into account that only 60% of the counted cells were neutrophils.

### Chemotaxis Assay:

IL-8 was diluted in HBSS containing 0.14 g/l CaCl₂ and 0.1 g/l MgSO₄ at concentrations of 10 μg/ml, 1 μg/ml and 0.1 μg/ml and put in the lower compartment of the chamber (26 μ1 per well). The freshly prepared neutrophils were seeded in the upper chamber (50 μl per well) and incubated for 30 minutes at 37° C. in a 5% CO₂ humidified incubator.

After incubation, the chamber was disassembled, the upper side of the heparin-coated filter was washed and wiped off and cells attached to the lower side were fixed with methanol and stained with Hemacolor solutions (Merck). Cells were then counted at 400× magnifications in 4 randomly selected microscopic fields per well. Finally, the mean of three independent experiments was plotted against the chemokine concentration.

### Preparation of Biotinylated Heparan Sulfate and Chondroitin Sulfate (GAG's)

GAG's are dissolved in 0.1 M MES buffer, pH 5.2. Then the solution is mixed with Biotin-LC-Hydrazide that was dissolved in DMSO to a final concentration of 50 mM. The weight ratio of GAG's to biotin-LC-hydrazide was 20:1. EDC, which has been dissolved in same buffer as GAG's, is added to the GAG solution to a final concentration of 6.5 mM. The labelling reaction takes place over 17 h at room temperature under gentle mixing the solution in an end-over motion. The reaction is stopped by dialysis against water over night.

Dialysis is carried out with a Spectra Por CE membrane with an MWCO of 500 Da.

Heparan sulfate and chondroitin sulfate were obtained from Celsus. Biotin-LC-Hydrazide and 1-Ethyl-3-[3-dimethylaminopropyl]carbodiimide Hydrochloride) (EDC) were purchased from Pierce. MES and DMSO were from Sigma Aldrich and Spectra Por Biotech Cellulose Ester (CE) membranes were purchased from Spectrum Laboratories Inc.

1. System to assess the biological activity of chemoattractants comprising at least a first unit and a second unit separated by a semipermeable carrier, characterized in that biologically active carbohydrate structures are immobilized on the surface of said carrier.

2. System according to claim 1 characterized in that the active carbohydrate structures are selected from the group of glycosaminoglycans (GAGs).

3. System according to claim 2 characterized in that the GAGs are naturally derived GAG structures, chemically synthesised GAG structures or chemically modified or substituted GAG structures.

4. System according to claim 2 characterized in that the GAGs are derived from, similar or identical to heparin, heparan sulfate, keratan sulfate, dermatan sulfate, chondroitin sulfate, and hyaluronic acid or any derivatives or fragments thereof.

5. System according to claim 1 characterized in that the chemoattractant is selected from the group consisting of chemokines, cytokines and growth factors.

6. System according to claim 1, characterized in that the chemokines are selected from the group consisting of IL-8, RANTES, SDF-1, I-TAC or MCP1 or derivatives or fragments thereof.

7. System according to claim 1, characterized in that the semipermeable carrier is a membrane, preferably selected from the group consisting of polycarbonate, polyvinyl, or polystyrene.

8. System according to claim 1, characterized in that the carbohydrate structure is non-covalently immobilized onto the membrane.

9. System according to claim 8 characterized in that non-covalent immobiliziation is via affinity binding, ionic/electrostatic interaction or hydrophobic interaction.

10. System according to claim 1 characterized in that the carbohydrate structure is covalently immobilized on the membrane, preferably via linker structures, preferably via aliphatic linkers, carbohydrate linkers, or aromatic compounds.

11. System according to claim 1 characterized in that the immobilized carbohydrate structure is GAG, preferably activated GAG, more preferably GAG activated by direct coupling via free primary amines, via acetyl groups, sulphate groups or hydroxyl groups.

12. System according to claim 1 characterized in that the first unit contains at least one chemoattractant optionally together with buffer and/or detergents.

13. System according to claim 12 characterized in that the ionic strength is >100 mM and the pH value is in the range of 6.5-7.5.

14. System according to claim 1 characterized in that the second unit contains at least one inhibitor of a chemoattractant and/or cells and/or media and/or buffer.

15. System according to claim 14 characterized in that the inhibitors of chemoattractants are selected from the group of GAGs, analogues, fragments or derivatives thereof and GAGmimetics.

16. System according to claim 15 characterized in that the inhibitors of chemoattractants are selected from the group of modified chemokines or mutant chemokines.

17. System to assess the biological activity of chemoattractants, comprising two units wherein the first unit contains chemokines, the second unit contains leukocytes, and wherein the units are separated by a semipermeable carrier having biotinylated heparin immobilized thereto

18. Use of a system according to claim 1 for measuring the degree of cell mobility.

19. Use of a system according to claim 1 for measuring the degree of chemotactic activity.
